(19) United States
(12) Patent Application Publication
(10) Pub. No.: US 2020/0155257 A1
(43) Pub. Date: May 21, 2020

(54) TREATMENT TOOL

(71) Applicant: OLYMPUS CORPORATION, Tokyo (JP)

(72) Inventor: Mitsuaki HASEGAWA, Tokyo (JP)

(73) Assignee: OLYMPUS CORPORATION, Tokyo (JP)

(21) Appl. No.: 16/773,244

(22) Filed: Jan. 27, 2020

Related U.S. Application Data
(63) Continuation of application No. PCT/JP2017/028902, filed on Aug. 9, 2017.

(51) Int. Cl. A61B 34/00; A61B 17/00

(52) U.S. Cl. CPC A61B 34/74; A61B 17/29; A61B 17/00234

(57) ABSTRACT

A treatment tool that includes an elongated shaft, a hollow socket connected to the shaft and having a first spherical surface with a constant radius, a ball inside the socket, and a wire extending through the shaft and causing a bending mechanism to bend. The rotation of the ball about a center point of the socket causes advancing and retreating of the wire. The ball includes a second spherical surface that is formed in a partial region of an outer surface of the ball, and that is configured to slide along the first spherical surface of the socket. The ball also includes a fixing portion attaching the wire to the ball, and a wire sliding surface on which the wire slides due to rotation of the ball.

### CROSS-REFERENCE TO RELATED APPLICATIONS

This is a continuation of International Application PCT/JP2017/028902 with an international filing date of Aug. 9, 2017, which is hereby incorporated by reference herein in its entirety.

### TECHNICAL FIELD

The present embodiments relate to a treatment tool.

### BACKGROUND

In the related art, there is a known treatment tool including a bending operation portion having a ball joint structure. The ball joint structure has a ball to which wires for driving a bending portion are connected, and a manipulating portion for rotating the ball.

### SUMMARY

An exemplary embodiment is a treatment tool including: (i) an elongated shaft having a bending mechanism attached to the shaft on a distal end side; (ii) a hollow socket connected to a base end of the shaft, the socket having a first spherical surface defining an inner surface of the socket, the first spherical surface having a constant radius with respect to a prescribed center point of the socket; (iii) a ball disposed and fitted inside the socket so as to be rotatable about the center point of the socket; and (iv) a wire connecting the bending mechanism and the ball, the wire extending through an inside of the shaft, the wire being configured to cause the bending mechanism to bend by advancing and retreating in a direction along a longitudinal axis of the shaft, and rotation of the ball about the center point of the socket is configured to cause the advancing and retreating of the wire. The ball includes: (i) a second spherical surface having a constant radius with respect to a center point of the ball, the second spherical surface being formed in a partial region of an outer surface of the ball, the radius of the second spherical surface of the ball being greater than a radius of the outer surface of the ball, such that the second spherical surface is configured to be slidable along the first spherical surface of the socket; (ii) a fixing portion attaching the wire to the ball; and (iii) a wire sliding surface located relatively closer to the distal end side of the shaft than the fixing portion of the ball in a direction along the longitudinal axis of the shaft, the wire sliding surface having a radius that is smaller than the radius of the second spherical surface and the radius of the outer surface of the ball, the wire being configured to slide along the wire sliding surface due to rotation of the ball, and the fixing portion is located between the second spherical surface and the wire sliding surface around a periphery of the ball. With such a ball joint structure, because the bending portion bends in a direction corresponding to the tilting direction of the manipulating portion, there is an advantage in that it is possible to intuitively perform the bending operation of the bending portion, and also to provide a bending operation portion by using a small number of components.

### BRIEF DESCRIPTION OF DRAWINGS

FIG. 1 is an overall configuration diagram showing a surgical system including a treatment tool according to an embodiment.

FIG. 2 is an overall configuration diagram showing the treatment tool according to the embodiment.

FIG. 3 is a configuration diagram showing a ball in a bending operation portion of the treatment tool in FIG. 2.

FIG. 4 is a longitudinal sectional view taken along a longitudinal axis of the ball in FIG. 3.

FIG. 5A is a diagram for explaining the positional relationship between a wire sliding surface of the ball and a wire when a manipulating handle is disposed at a neutral position.

FIG. 5B is a diagram for explaining the positional relationship between the wire sliding surface of the ball and the wire when the manipulating handle is tilted in the rightward direction.

FIG. 6A is a diagram for explaining the movement of a protrusion in a groove when the ball is rotated in the leftward direction.

FIG. 6B is a diagram for explaining the movement of the protrusion in the groove when the ball is rotated in the rightward direction.

FIG. 6C is a diagram for explaining the movement of the protrusion in the groove when the ball is rotated in the upward direction.

FIG. 7 is a longitudinal sectional view of the ball showing a modification of arrangement of the grooves and the protrusions.

FIG. 8 is a diagram showing a modification of a manipulating portion of the treatment tool in FIG. 2.

FIG. 9 is a diagram showing a modification of the ball shown in FIG. 3.

### DESCRIPTION

A treatment tool 1 according to a present embodiment will be described below with reference to the drawings.

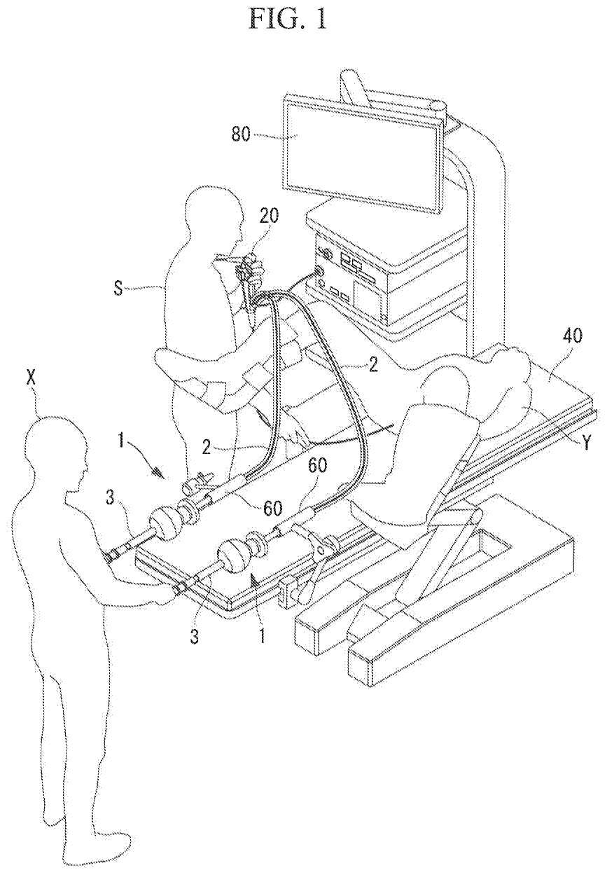

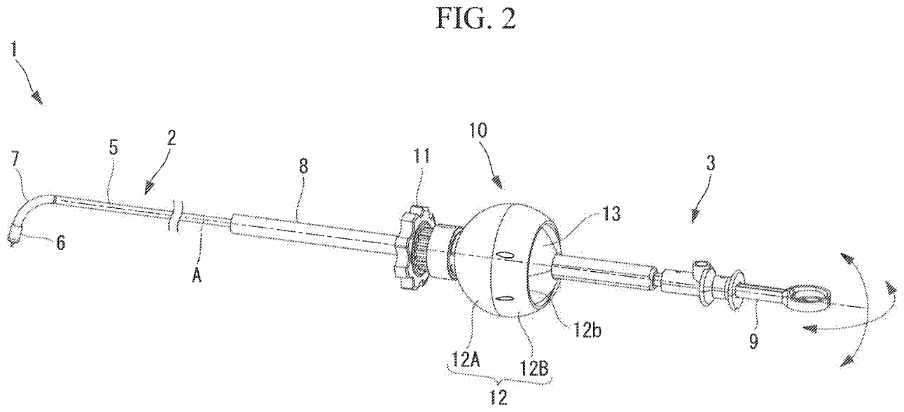

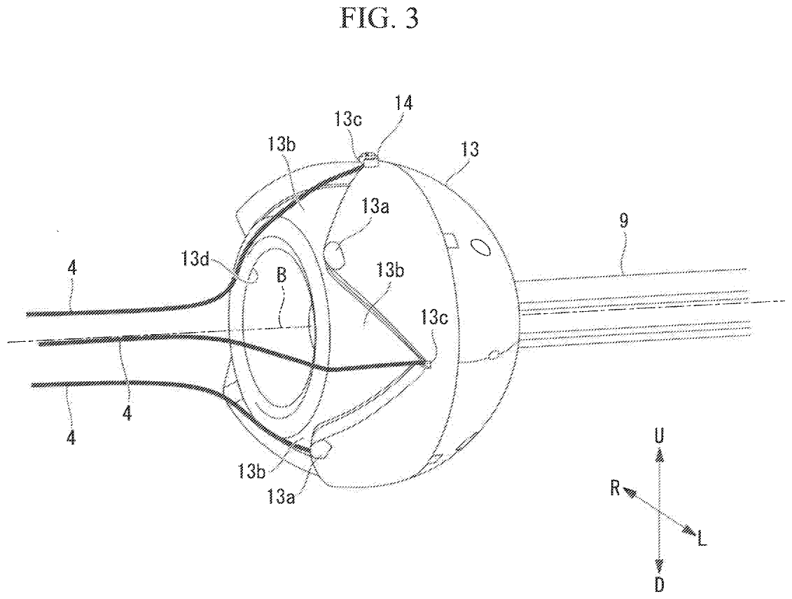

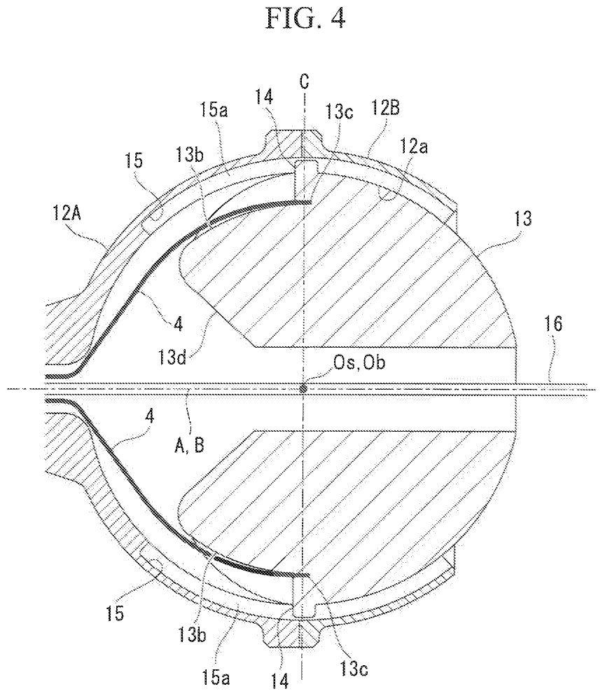

As shown in FIGS. 1 and 2, the treatment tool 1 according to this embodiment includes an elongated shaft 2 that has a bending portion (bending mechanism) 7 on the distal end side and that is inserted into a body, a manipulating portion 3 that is connected to the base end of the shaft 2 and that is manually operated by an operator X, and wires 4 (see FIGS. 3 and 4) that connect the bending portion 7 and the manipulating portion 3 through the inside of the shaft 2 and that cause the bending portion 7 to bend in accordance with the operation performed at the manipulating portion 3.

FIG. 1 is an overall configuration diagram showing a surgical system including the treatment tool 1 according to this embodiment. As shown in FIG. 1, the surgical system includes an endoscope 20 operated by a scopist S, the treatment tool 1 operated by the operator X, a treatment-tool holder 60 that is fixed to a bed 40 on which a patient Y lies and that supports the treatment tool 1, and a display 80 that displays an endoscope image observed with the endoscope 20. The treatment-tool holder 60 is a tubular member through which the shaft 2 penetrates. The shaft 2 is inserted into the body of the patient Y via a treatment-tool channel provided in the endoscope 20 or via a channel externally attached to the endoscope 20. FIG. 1 shows an example in which the endoscope 20 has two treatment-tool channels, and the operator X operates two treatment tools 1 with both hands. The operator X can manipulate the position and orientation of an end effector provided at the distal end of the shaft 2 by operating the manipulating portion 3 disposed outside of the body, while observing the end effector with the endoscope 20.

As shown in FIG. 2, the shaft 2 includes an elongated flexible portion 5, a distal end portion 6 that is disposed on the distal end side of the flexible portion 5, and the bending portion 7 that connects the flexible portion 5 and the distal end portion 6. The bending portion 7 is capable of bending in a direction intersecting a longitudinal axis A of the flexible portion 5. The distal end portion 6 is provided with an end effector (for example, forceps or a knife) for treating biological tissue.

The wires 4 are arranged in the flexible portion 5 in a direction along the longitudinal axis A. Distal end portions of the wires 4 are fixed to the bending portion 7, and base end portions of the wires 4 are fixed to the manipulating portion 3. The four wires 4 respectively corresponding to the upper, lower, left, and right sides of the bending portion 7 are arranged at substantially equal intervals in a circumferential direction about the longitudinal axis A of the flexible portion 5. The up-down direction and the left-right direction of the bending portion 7 are directions that are individually orthogonal to the longitudinal axis A of the flexible portion 5 and that are also orthogonal to each other. The individual wires 4 are capable of advancing and retreating in the direction along the longitudinal axis A of the flexible portion 5, and the bending portion 7 bends in a direction corresponding to a wire 4 that retreats to the base end side.

The manipulating portion 3 includes a rigid rotary shaft 8 that is connected to the base end of the flexible portion 5 and that extends coaxially with the flexible portion 5, a manipulating handle 9 that is disposed on the base end side of the rotary shaft 8 and that is gripped by the operator X, and a bending operation portion 10 and a rotating operation portion 11 that are provided between the rotary shaft 8 and the manipulating handle 9. The bending operation portion 10 is used for performing the bending operation of the bending portion 7. The rotating operation portion 11 is used for rotating the shaft 2 about the longitudinal axis A with respect to the manipulating handle 9 and the bending operation portion 10.

The bending operation portion 10 has a ball joint structure including a substantially spherical hollow socket 12 that is connected to the rotary shaft 8 via the rotating operation portion 11, and a substantially spherical ball 13 that is rotatably fitted inside the socket 12.

The socket 12 is composed of two substantially hemispherical hollow members 12A, 12B that are coupled to each other by means of coupling members, such as screws. As shown in FIG. 4, an inner surface (first spherical surface) 12a of the socket 12 is a spherical surface having a constant radius with respect to a prescribed center point Os. In FIG. 4, the illustration of some structures is omitted. The socket 12 as a whole has a shape in which a portion on the base end side of the sphere is cut out in a plane orthogonal to the longitudinal axis A of the shafts 2, 8, and an opening 12b is provided on the base end surface thereof. As described above, because the socket 12 has a spherical shape larger than a hemisphere, the ball 13 is held in the socket 12 without falling out of the opening 12b.

The ball 13 is supported on the inner surface 12a of the socket 12 such that a prescribed center point Ob of the ball 13 coincides with the center point Os of the socket 12, and is rotatable in any direction about the center points Os, Ob with respect to the socket 12. The manipulating handle 9 has a substantially straight rod shape, is connected to an outer surface of the ball 13 that is exposed to the outside of the socket 12 from the opening 12b, and extends to the opposite side from the rotary shaft 8 and the socket 12. As shown in FIG. 2, the manipulating handle 9 is aligned with the shafts 2, 8 at a neutral position, and is tilted in an arbitrary direction about the center points Os, Ob by means of rotation of the ball 13 in the socket 12.

The four wires 4 led out of the base end of the flexible portion 5 pass through the interior of the rotary shaft 8 and the rotating operation portion 11, extend into the socket 12, and are arranged between the inner surface 12a of the socket 12 and the outer surface of the ball 13. In a state in which the manipulating handle 9 is disposed at the neutral position, the base end portions of the four wires 4 are fixed on the outer surface of the ball 13 at positions (fixing portions 13c) equally spaced in the circumferential direction about the longitudinal axis A.

The operator X rotates the ball 13 in the socket 12 about the center points Os, Ob by tilting the manipulating handle 9 in a direction intersecting the longitudinal axis A from the neutral position, and by doing so, it is possible to pull the wire 4 corresponding to the tilting direction of the manipulating handle 9, thereby bending the bending portion 7. For example, when the manipulating handle 9 is tilted in the rightward direction, the left wire 4 is pulled and the right wire 4 is pushed out by the rightward-direction rotation of the ball 13, whereby the bending portion 7 is bent to the left. When doing so, the operator X can tilt the manipulating handle 9 to a prescribed maximum tilting angle at which the manipulating handle 9 abuts against an edge of the opening 12b of the socket 12. In other words, the edge of the opening 12b functions as a limiter for restricting the rotation angle of the ball 13 in the socket 12 within a prescribed angular range.

Next, the structure of the ball 13 will be described in more detail.

The ball 13 has a prescribed central axis B that passes through the center point Ob and that is aligned with the longitudinal axis A of the shafts 2, 8 in a state in which the manipulating handle 9 is disposed at the neutral position. As shown in FIGS. 3 and 4, the ball 13 as a whole has a shape in which a portion on the distal end side of the sphere is cut out in a plane orthogonal to the central axis B. The outer surface of the ball 13 has a stepped shape having a socket sliding surface (second spherical surface) 13a that comes into contact with the inner surface 12a of the socket 12 and that slides along the inner surface 12a of the socket 12, and a wire sliding surface 13b that is offset more radially inward than the socket sliding surface 13a and on which the wire 4 is arranged.

In the outer surface of the ball 13, the socket sliding surface 13a is a region having the maximum diameter and is formed of a portion of a spherical surface having a constant radius substantially equal to that of the inner surface 12a of the socket 12, with respect to the prescribed center point Ob of the ball 13. The wire sliding surface 13b is formed of a portion of a spherical surface having a constant radius smaller than the radius of the socket sliding surface 13a, with respect to the center point Ob, and has a substantially triangular shape (substantially spherical triangular shape) having one apex positioned on the base end side and gradually widening toward the distal end side.

Four wire sliding surfaces 13b are uniformly provided in a circumferential direction about the central axis B so that one wire sliding surface 13b corresponds to one wire 4. The socket sliding surface 13a is formed in a triangular region sandwiched by two wire sliding surfaces 13b adjacent in the circumferential direction. The ball 13 is supported on the inner surface 12a of the socket 12, at the four socket sliding surfaces 13a, thereby being fitted inside the socket 12 such that the center point Ob of the ball 13 coincides with the center point Os of the socket 12.

The fixing portions 13c to which the base end portions of the wires 4 are fixed are provided at the apex positions on the base end side of the respective wire sliding surfaces 13b. The fixing portions 13c are, for example, holes into which the base end portions of the wires 4 are inserted. The difference between the radius of the socket sliding surface 13a and the radius of the wire sliding surface 13b is larger than the diameter of the wire 4. Therefore, in a state in which the ball 13 is fitted inside the socket 12, a gap larger than the diameter of the wire 4 is formed between the inner surface 12a of the socket 12 and the wire sliding surface 13b, which is closer to the distal end side than the fixing portion 13c is, and the wire 4 can be smoothly moved in the gap.

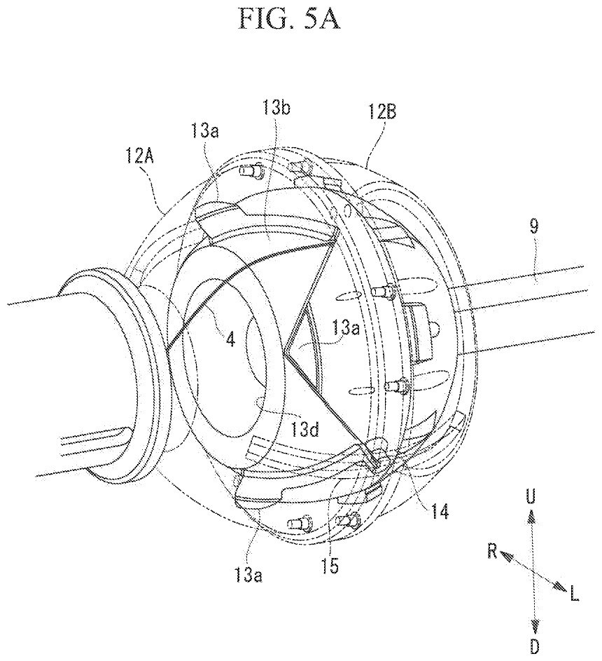

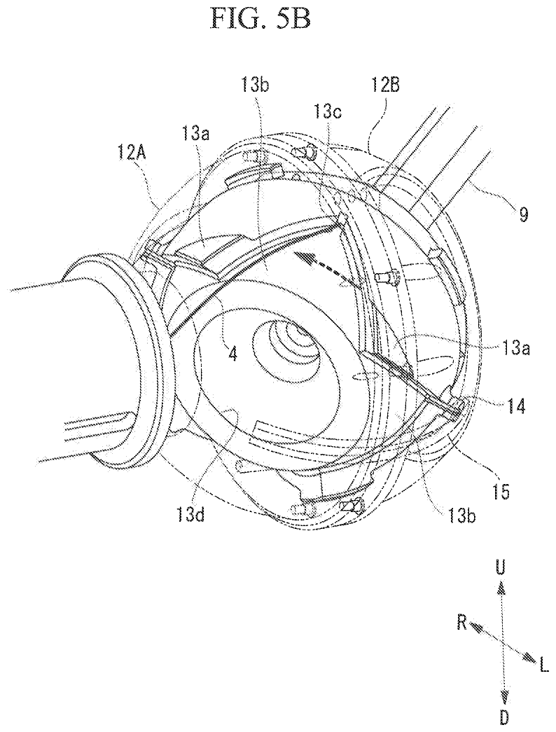

FIGS. 5A and 5B show the movement of a wire 4 by means of rotation of the ball 13. When the ball 13 is rotated, the wire 4 extending in the rotating direction of the ball 13 is pulled or pushed by the ball 13. Meanwhile, regarding the wire 4 extending in a direction orthogonal to the rotating direction of the ball 13, as shown in FIGS. 5A and 5B, only the wire sliding surface 13b slides in a direction orthogonal to the longitudinal direction of the wire 4, and the wire 4 maintains the position thereof without being pulled or pushed. Among the upper, lower, left, and right wires 4, only the upper wire 4 is shown in FIGS. 5A and 5B.

Here, the dimensions of the wire sliding surface 13b in the circumferential direction are designed so that, when the ball 13 is rotated to the maximum angle in the prescribed angular range restricted by the limiter, an end of the wire sliding surface 13b in the circumferential direction is disposed at a position separated from the wire 4 without reaching the wire 4. Specifically, the apex angle of the wire sliding surface 13b is designed in accordance with the prescribed maximum tilting angle of the manipulating handle 9, which is restricted by the limiter.

As shown in FIG. 4, the ball 13 has a through-hole 13d penetrating therethrough along the central axis B and into which a wire 16 for driving an end effector is inserted. The through-hole 13d has a truncated cone shape in which the diameter thereof gradually increases toward the distal end side. As with the apex angle of the socket sliding surface 13a, the center angle of the through-hole 13d is determined in accordance with the prescribed maximum tilting angle of the manipulating handle 9, which is restricted by the limiter. By doing so, when the ball 13 is rotated, the wire 16 is prevented from coming into contact with an inner surface of the through-hole 13d and being unintendedly pulled, and thus, the operation of the end effector is prevented from being affected by the tilting operation of the manipulating handle 9.

Next, the operation of the thus-configured treatment tool 1 will be described.

With the treatment tool 1 according to this embodiment, when the operator X tilts the manipulating handle 9, for example, as shown in FIG. 5B, in the rightward direction, the ball 13 in the socket 12 is rotated in the rightward direction, whereby the left wire 4 is pulled to the base end side, the right wire 4 is pushed out to the distal end side, and the bending portion 7 is bent to the left. In this case, because the upper and lower wires 4 are not pulled or pushed by the ball 13, the angle of the bending portion 7 in the up-down direction does not change. As described above, because only the wires 4 corresponding to the tilting direction of the manipulating handle 9 selectively advance and retreat, it is possible to control the bending direction of the bending portion 7 by the tilting direction of the manipulating handle 9.

In this case, with this embodiment, the wire 4 is arranged on the wire sliding surface 13b, which is offset radially inward relative to the socket sliding surface 13a in the outer surface of the ball 13. With this configuration, the wire 4 is prevented from getting caught in between the inner surface 12a of the socket 12 and the socket sliding surface 13a, which slide against each other, and from being dragged by the rotating ball 13. As a result, it is possible to prevent an unintended wire 4 from being pulled and to control the bending direction of the bending portion 7 in a direction correctly corresponding to the tilting direction of the manipulating handle 9. In addition, the dimensions of the wire sliding surface 13b are designed so that the wire 4 is positioned on the wire sliding surface 13b even in a state in which the ball 13 is rotated to the maximum angle. By doing so, it is possible to prevent the wire 4 from getting caught in between the inner surface 12a of the socket 12 and the socket sliding surface 13a in a more reliable manner.

In addition, because the wire 4 pulled by the rotating ball 13 bends along the wire sliding surface 13b, the traction amount of the wire 4 also depends on the shape of the wire sliding surface 13b in addition to the rotation angle of the ball 13. With this embodiment, because the wire sliding surface 13b has a spherical shape concentric with the ball 13, the traction amount of the wire 4 linearly changes with respect to the rotation angle of the ball 13. Therefore, it is possible to easily control the bending angle of the bending portion 7 by the rotation angle of the ball 13. However, the wire sliding surface 13b may have a shape other than a spherical surface, for example, a polygonal surface centered on the center point Ob.

In addition, in order to make the socket sliding surface 13a smoothly slide along the inner surface 12a of the socket 12, a high precision is required for machining the socket sliding surface 13a. With this embodiment, there is an advantage in that the machining of the ball 13 is facilitated by providing the socket sliding surface 13a in a narrow region sandwiched between the two wire sliding surfaces 13b, thus reducing the area of the socket sliding surface 13a.

In this embodiment, as shown in FIG. 4, it is preferable that a pair of protrusions 14 be provided on the outer surface of the ball 13 and that a pair of grooves 15 be provided in the inner surface 12a of the socket 12, so that rotation of the ball 13 about the longitudinal axis A with respect to the socket 12 is prevented. In a case in which the ball 13 in the socket 12 is rotatable about the longitudinal axis A, the correspondence relationship between the tilting direction of the manipulating handle 9 and the bending direction of the bending portion 7 changes in accordance with the rotation angle of the ball 13 about the longitudinal axis A, and thus, it becomes difficult to intuitively operate the bending portion 7. By preventing such a rotation of the ball 13 about the longitudinal axis A by means of the protrusions 14 and the grooves 15, it is possible to maintain the intuitive operability of the bending portion 7.

The pair of protrusions 14 have a columnar shape protruding radially outward from the outer surface of the ball 13. The pair of protrusions 14 are provided at positions facing each other in a radial direction intersecting the central axis B (preferably, orthogonal to the central axis B), with the center point Ob of the ball 13 interposed therebetween, and central axes C of the pair of protrusions 14 are arranged on the same straight line passing through the center point Ob of the ball 13.

Each of the grooves 15 has a pair of side walls 15a that face each other in the circumferential direction about the longitudinal axis A, with a gap substantially equal to the diameter of the protrusion 14, and that extend in a direction orthogonal to the circumferential direction about the longitudinal axis A. Only one of the pair of side walls 15a is shown in FIG. 4. The pair of grooves 15 are provided at positions facing each other in a radial direction intersecting the longitudinal axis A, so as to correspond to the movable ranges of the pair of protrusions 14, which are associated with rotation of the ball 13.

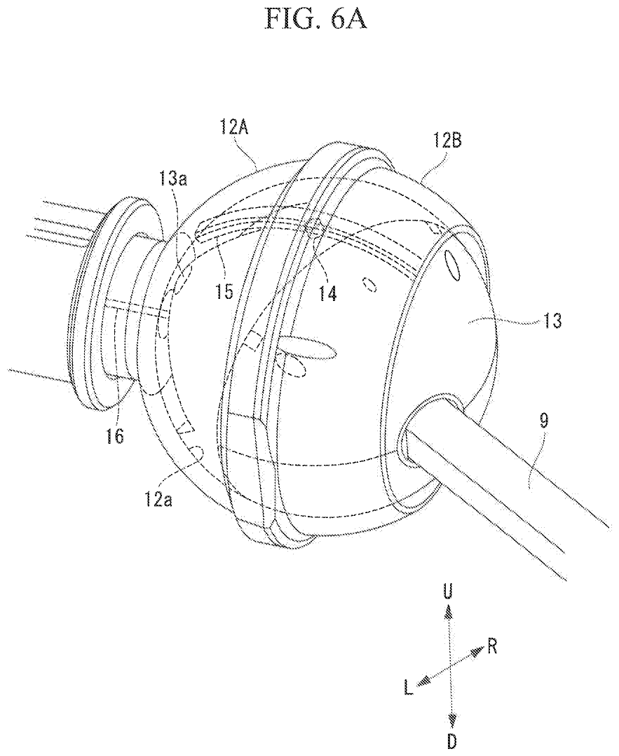

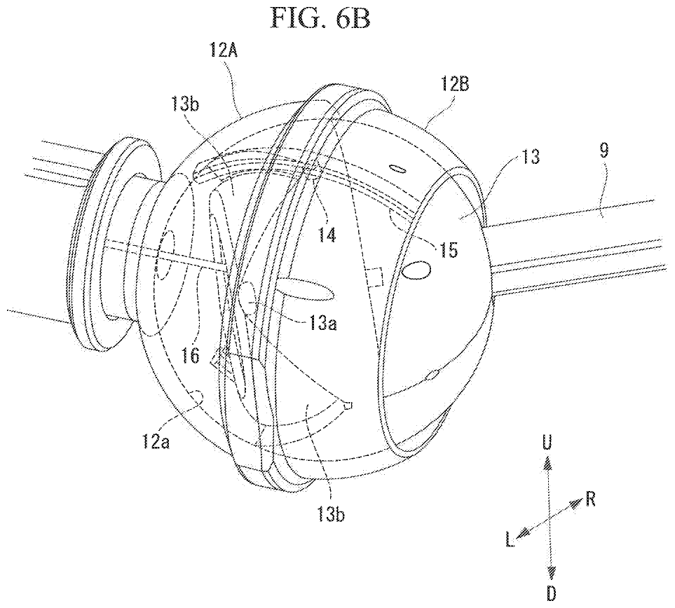

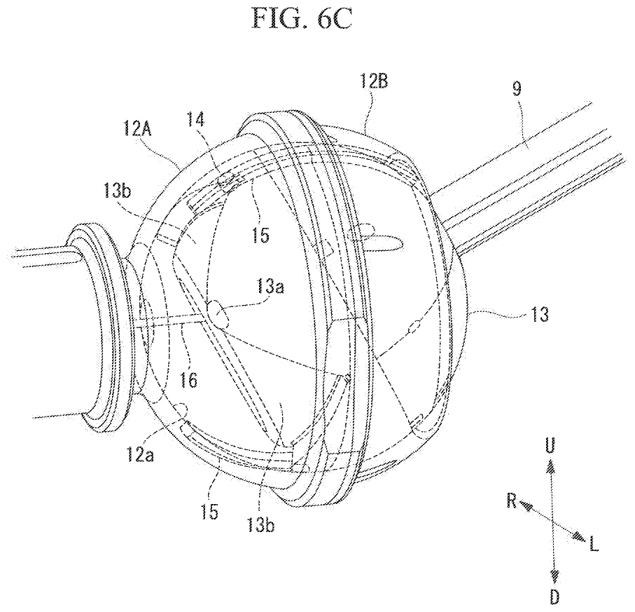

The protrusion 14 is inserted between the pair of side walls 15a, is rotatable about the central axis C in between the pair of side walls 15a, and is also movable in a direction orthogonal to the circumferential direction about the longitudinal axis A in between the pair of side walls 15a. Therefore, rotation of the ball 13 about the longitudinal axis A is prevented by the protrusion 14 that abuts against the side walls 15a and the side walls 15a stopping movement of the protrusion 14 in the circumferential direction about the longitudinal axis A; however, other rotations of the ball 13 about the center points Os, Ob are allowed by movement, rotation, or a combination of movement and rotation of the protrusion 14 in the groove 15. Specifically, as shown in FIGS. 6A and 6B, the ball 13 is rotated in the left (L) direction and the right (R) direction by the rotation of the protrusion 14 about the central axis C, and as shown in FIG. 6C, the ball 13 is rotated in the upward (U) direction and the downward (D) direction by the movement of the protrusion 14 in the groove 15.

The grooves 15 are formed at least in the inner surface of the distal-end-side member 12A so that at least one of the protrusions 14 is always positioned in the groove 15 regardless of the rotation angle of the ball 13, and preferably, the grooves 15 are also formed in the inner surface of the base-end-side member 12B so that the respective protrusions 14 are always positioned in the grooves 15.

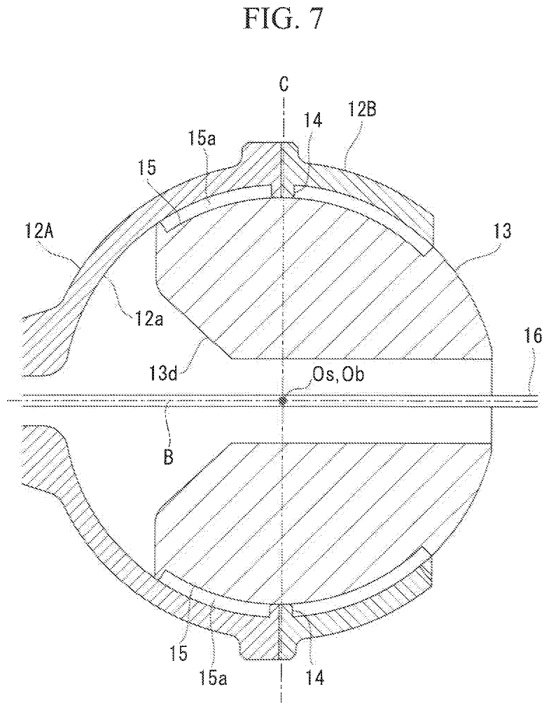

As shown in FIG. 7, the grooves 15 may be formed in the outer surface of the ball 13, and the protrusions 14 may be provided on the inner surface 12a of the socket 12. Even if the arrangement of the grooves 15 and the protrusions 14 is reversed in this way, among rotations of the ball 13 about the prescribed center points Os, Ob, rotation of the ball 13 about the longitudinal axis A can be prevented, while rotations in other directions can be allowed.

It is preferable that the protrusion 14 have a columnar shape or spherical shape in which a cross section orthogonal to the central axis C forms a perfect circle, so that the clearance between the side walls 15a and the protrusion 14 is constant regardless of the rotation angle of the protrusion 14 about the central axis C in the groove 15. However, the protrusion 14 may have another shape, such as a prismatic shape. In addition, only one each of the protrusion 14 and the groove 15 may be provided. In this case, it is preferable that the length of the groove 15 and the maximum tilting angle of the manipulating handle 9 be designed so that the protrusion 14 does not come out of the groove 15 even in a state in which the manipulating handle 9 is tilted to the maximum tilting angle.

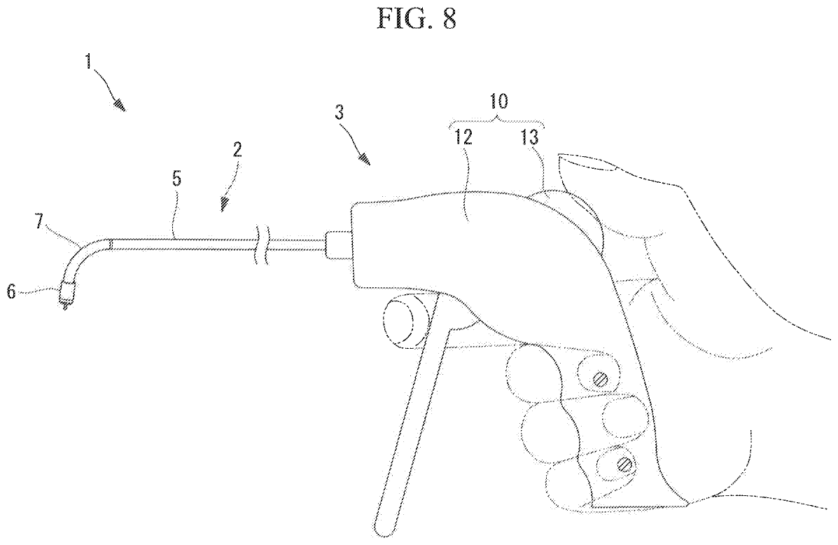

Although the ball 13 is rotated by performing the tilting operation of the manipulating handle 9 in this embodiment, alternatively, the ball 13 may be rotated by performing another operation. For example, as shown in FIG. 8, a trackball-type bending operation portion 10 in which the ball 13 is directly manipulated with the thumb of the operator X may be employed. The wiring path of the wire 16 for driving an end effector differs depending on the structure of the manipulating portion 3. In a case in which the wire 16 does not pass through the ball 13, the through-hole 13d is not necessary and may be omitted.

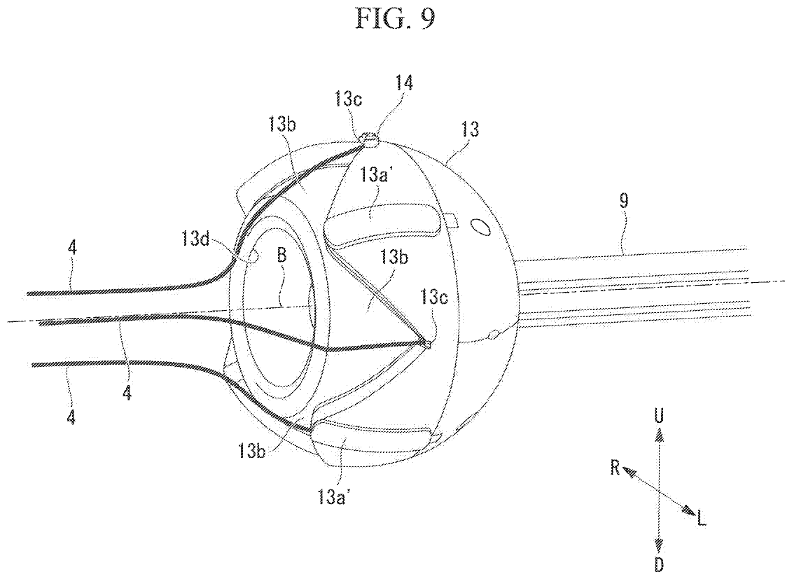

Although the socket sliding surfaces (second spherical surface) 13a of the ball 13 shown in FIG. 3 are respectively formed in the three triangular distal-end-side partial regions each of which is sandwiched by two wire sliding surfaces 13b adjacent in the circumferential direction, as shown in FIG. 9, socket sliding surfaces (second spherical surface) 13a′ of the ball 13 may be formed so as to extend from the aforementioned triangular distal ends to regions beyond the fixing portions 13c in a direction orthogonal to the circumferential direction about the longitudinal axis. By doing so, the backlash of the ball 13 in the socket 12 is reduced, making it possible to enhance the sliding stability. Although the socket sliding surfaces 13a′ are provided at three locations on the outer surface of the ball 13 in FIG. 9, more of them may be provided.

As a result, the following aspect is read from the above described embodiment:

A treatment tool including: (i) an elongated shaft having a bending mechanism attached to the shaft on a distal end side; (ii) a hollow socket connected to a base end of the shaft, the socket having a first spherical surface defining an inner surface of the socket, the first spherical surface having a constant radius with respect to a prescribed center point of the socket; (iii) a ball disposed and fitted inside the socket so as to be rotatable about the center point of the socket; and (iv) a wire connecting the bending mechanism and the ball, the wire extending through an inside of the shaft, the wire being configured to cause the bending mechanism to bend by advancing and retreating in a direction along a longitudinal axis of the shaft, and rotation of the ball about the center point of the socket is configured to cause the advancing and retreating of the wire. The ball includes: (i) a second spherical surface having a constant radius with respect to a center point of the ball, the second spherical surface being formed in a partial region of an outer surface of the ball, the radius of the second spherical surface of the ball being greater than a radius of the outer surface of the ball, such that the second spherical surface is configured to be slidable along the first spherical surface of the socket; (ii) a fixing portion attaching the wire to the ball; and (iii) a wire sliding surface located relatively closer to the distal end side of the shaft than the fixing portion of the ball in a direction along the longitudinal axis of the shaft, the wire sliding surface having a radius that is smaller than the radius of the second spherical surface and the radius of the outer surface of the ball, the wire being configured to slide along the wire sliding surface due to rotation of the ball, and the fixing portion is located between the second spherical surface and the wire sliding surface around a periphery of the ball.

With this aspect, when the ball in the socket is rotated about the prescribed center point, the wire extending in the rotating direction of the ball is pulled to retreat or is pushed to advance, whereby the bending mechanism is bent. Therefore, it is possible to bend the bending mechanism in a direction corresponding to the rotating direction of the ball. In this case, the outer surface of the ball has a stepped shape having the second spherical surface that slides along the first spherical surface, which is the inner surface of the socket, and the wire sliding surface that is offset more radially inward than the second spherical surface, and the wire is arranged on the wire sliding surface. With this configuration, the wire is prevented from getting caught in between the first spherical surface and the second spherical surface that slide against each other; therefore, it is possible to prevent unintended traction of the wire, thus ensuring an intuitive operability.

In the abovementioned aspect, the second spherical surface may be formed in a region sandwiched by two wire sliding surfaces adjacent in a circumferential direction. In the abovementioned aspect, the difference between the radius of the wire sliding surface and the radius of the second spherical surface may be larger than a diameter of the wire. In the abovementioned aspect, the difference between a diameter of the first spherical surface and a diameter of the second spherical surface may be smaller than the diameter of the wire. In the abovementioned aspect, the second spherical surface may be provided at a plurality of locations on the outer surface of the ball.

In the abovementioned aspect, a limiter for restricting a rotation angle of the ball in the socket within a prescribed angular range may be provided, and an end of the wire sliding surface in the circumferential direction about the longitudinal axis may be disposed at a position separated from the wire in a state in which the ball is rotated to a maximum angle within the prescribed angular range. With respect to the wire extending in a direction intersecting the rotating direction of the ball, the wire sliding surface of the ball slides in a direction intersecting a longitudinal direction of the wire. In this case, because the wire is positioned on the wire sliding surface even in a state in which the ball is rotated to the maximum angle restricted by the limiter, the wire is prevented from interfering with a surface other than the wire sliding surface. With this configuration, it is possible to prevent unintended traction of the wire in a more reliable manner.

In the abovementioned aspect, a protrusion that is provided on one of the outer surface of the ball and the inner surface of the socket, and a groove that is provided in the other of the outer surface of the ball and the inner surface of the socket and that has a pair of side walls that are mutually separated in the circumferential direction about the longitudinal axis may be provided, the protrusion may be inserted between the pair of side walls, and movement thereof in the circumferential direction may be stopped by the pair of side walls. With this configuration, rotation of the ball about the longitudinal axis of the shaft is prevented by the protrusion abutting against the side walls, and an orientation of the ball about the longitudinal axis with respect to the bending mechanism is kept constant. This prevents the correspondence relationship between the rotating direction of the ball and the bending direction of the bending mechanism from changing during use, and it is possible to maintain the intuitive operability of the bending mechanism by means of rotation of the ball.

In the abovementioned aspect, the pair of side walls may extend in a direction orthogonal to the circumferential direction about the longitudinal axis, and the protrusion may be rotatable about an axis passing through the protrusion and the center point in between the pair of side walls and may also be movable in the aforementioned orthogonal direction in between the pair of side walls. With this configuration, rotation of the ball about the longitudinal axis of the shaft can be prevented, while rotations of the ball other than the rotation about the longitudinal axis can be allowed.

In the abovementioned aspect, a pair of the protrusions may be provided on said one of the outer surface of the ball and the inner surface of the socket, at positions facing each other in a radial direction, with the center point interposed therebetween, and a pair of the grooves may be provided in said other of the outer surface of the ball and the inner surface of the socket, at positions facing each other in a direction intersecting the longitudinal axis, with the longitudinal axis interposed therebetween. When the ball is rotated, the pair of protrusions move in the grooves in mutually opposite directions. Therefore, by providing the pair of grooves at least in a distal-end-side half or a base-end-side half of the movable ranges of the protrusions, it is possible to position one of the protrusions in the groove, even if the other protrusion comes out of the groove. In other words, even in a case in which the lengths of the grooves are smaller, either one of the pair of protrusions can be positioned in the groove; thus, it is possible to prevent rotation of the ball about the longitudinal axis of the shaft.

In the abovementioned aspect, the wire sliding surface may be a spherical surface having a constant radius with respect to the center point. Because the wire pulled by means of rotation of the ball bends along the wire sliding surface, the traction amount of the wire also depends on the shape of the wire sliding surface in addition to the rotation angle of the ball. By configuring the wire sliding surface so as to have a spherical shape concentric with the ball, the traction amount of the wire linearly changes with respect to the rotation angle of the ball; thus, it is possible to control the bending angle of the bending mechanism by the rotation angle of the ball.

### REFERENCE SIGNS LIST

1 treatment tool
2 shaft
3 manipulating portion
4 wire
5 flexible portion
6 distal end portion
7 bending portion (bending mechanism)
8 rotary shaft
9 manipulating handle
10 bending operation portion
11 rotating operation portion
12 socket
12a inner surface (first spherical surface)
12b opening (limiter)
13 ball
13a socket sliding surface (second spherical surface)
13b wire sliding surface
13c fixing portion
13d through-hole
14 protrusion
15 groove
16 wire
20 endoscope
40 bed
60 treatment-tool holder
80 display

1. A treatment tool comprising:
an elongated shaft having a bending mechanism attached to the shaft on a distal end side;
a hollow socket connected to a base end of the shaft, the socket having a first spherical surface defining an inner surface of the socket, the first spherical surface having a constant radius with respect to a prescribed center point of the socket;
a ball disposed and fitted inside the socket so as to be rotatable about the center point of the socket; and
a wire connecting the bending mechanism and the ball, the wire extending through an inside of the shaft, the wire being configured to cause the bending mechanism to bend by advancing and retreating in a direction along a longitudinal axis of the shaft, and rotation of the ball about the center point of the socket is configured to cause the advancing and retreating of the wire,
wherein the ball includes:
a second spherical surface having a constant radius with respect to a center point of the ball, the second spherical surface being formed in a partial region of an outer surface of the ball, the radius of the second spherical surface of the ball being greater than a radius of the outer surface of the ball, such that the second spherical surface is configured to be slidable along the first spherical surface of the socket,
a fixing portion attaching the wire to the ball, and
a wire sliding surface located relatively closer to the distal end side of the shaft than the fixing portion of the ball in a direction along the longitudinal axis of the shaft, the wire sliding surface having a radius that is smaller than the radius of the second spherical surface and the radius of the outer surface of the ball, the wire being configured to slide along the wire sliding surface due to rotation of the ball, and the fixing portion is located between the second spherical surface and the wire sliding surface around a periphery of the ball.

2. The treatment tool according to claim 1, wherein the second spherical surface is formed in a region of the ball that is sandwiched between two wire sliding surfaces adjacent to each other in a circumferential direction of the ball, the two wire sliding surfaces including the wire sliding surface.

3. The treatment tool according to claim 1, wherein a difference between the radius of the wire sliding surface and the radius of the second spherical surface is greater than a diameter of the wire.

4. The treatment tool according to claim 1, wherein a difference between a diameter of the first spherical surface and a diameter of the second spherical surface is smaller than a diameter of the wire.

5. The treatment tool according to claim 1, wherein a plurality of second spherical surfaces, which includes the second spherical surface, are respectively provided at a plurality of locations on the outer surface of the ball.

6. The treatment tool according to claim 1, further comprising a limiter configured to restrict a rotation angle of the ball in the socket within a prescribed angular range,
wherein an end of the wire sliding surface in a circumferential direction about the longitudinal axis is disposed at a position separated from the wire when the ball is rotated to a maximum angle within the prescribed angular range.

7. The treatment tool according to claim 1, further comprising:
a protrusion protruding from one of the outer surface of the ball and the inner surface of the socket; and
a groove provided in another one of the outer surface of the ball and the inner surface of the socket, the groove having a pair of side walls that are mutually separated in a circumferential direction about the longitudinal axis,
wherein the protrusion is inserted between the pair of side walls, and movement of the protrusion in the circumferential direction is stopped by the pair of side walls.
